(19) United States
(12) Patent Application Publication
(10) Pub. No.: US 2004/0049204 A1
(43) Pub. Date: Mar. 11, 2004

(54) System and corresponding method for deploying an implantable intraluminal device

(76) Inventors: Eran Harari, Doar Na Menashe (IL); Ygael Grad, Tel Aviv (IL); Ofer Yodfat, Modi'in (IL)

Correspondence Address: Anthony Castorina, G E Ehrlich, Suite 207, 2001 Jefferson Davis Highway, Arlington, VA, 22202, US

(21) Appl. No.: 10/451,642

(22) PCT Filed: Jan. 11, 2002
(86) PCT No.: PCT/IL02/00023

(30) Foreign Application Priority Data
Jan. 11, 2001 (IL) 140870

(51) Int. Cl. A61F 11/00; A61F 2/06

(52) U.S. Cl. 606/108; 623/1.11

(57) ABSTRACT

A system for delivering and for deploying an expandable intraluminal device at a selected location in a body lumen wall, includes: an inner supporting structure for supporting the intraluminal device; an outer tube for enclosing the inner supporting structure, and the intraluminal device when supported in its contracted condition thereon; and a pair of socks for receiving between them the ends of the intraluminal device in its contracted condition. Each sock has one end secured to the inner supporting structure, an opposite end facing the other sock for receiving the respective end of the intraluminal device in its contracted condition, and a length such that, when the outer tube is removed and the intraluminal device starts to expand, each sock does not release the respective end of the intraluminal device until the center region of the intraluminal device expands into engagement with the body lumen wall.

### FIELD AND BACKGROUND OF THE INVENTION

The present invention relates to implantable medical device delivery systems and, more particularly, to a system and corresponding method for optimally deploying an implantable expandable intraluminal device at the required location in a body lumen.

The use of balloon angioplasty catheters for the dilation of various vessels of the human body and most particularly for opening stenotic arteries is well known, as is the placement of stents into vessels to retain their patency. Uses of balloon catheters for expanding expandable stents mounted on them, as well as their use for embedding a stent into a vessel wall to prevent stent migration are equally well known in the art.

For balloon expandable stents, the deployment procedure combines the placement of the stent in position and embedding of it into a vessel wall. Using self-expanding stents, it is typical to use separate catheters for vessel dilatation, stent delivery, and post delivery dilation, to secure the stent in position and embed it in the vessel wall. This requires one or more catheter exchanges, which increases time and cost for performing interventional procedures.

Many solutions for integrating a balloon catheter and a stent delivery catheter into a single device, capable of performing balloon angioplasty and stent delivery, are taught about in the prior art. For example, in U.S. Pat. No. 5,019,090, there is described a method for mounting a self-deploying stent on a balloon angioplasty catheter. The balloon, located at the distal end of the catheter, is expanded to open the stenotic zone and then deflated. The catheter is then moved distally to position the stent, which was mounted on the catheter proximally to the balloon, within the lesion. A restraining sheath is withdrawn and the stent is allowed to expand. The catheter is then moved proximally until the balloon is inside the partially expanded stent, the balloon is again expanded to complete expansion and anchoring of the stent. U.S. Pat. No. 5,192,297 describes a similar system, except that here, the stent is located at the distal end and allowed to partially expand first. Then, the catheter is advanced distally to position the balloon within the stent where it is expanded to complete the placement of the stent. U.S. Pat. No. 5,634,928 teaches about an integrated coaxial system consisting of an inner balloon catheter and an outer catheter that slides over the inner catheter and contains a stent. The balloon is first expanded to dilate the vessel and then deflated. The catheter containing the stent is then advanced distally to position the stent over the balloon where it is released. The outer catheter is then moved proximally and the balloon is expanded again to complete deployment of the stent.

In all three of these teachings, the balloon and the stent are displaced laterally on the catheter which must be moved backwards and forwards in order to accomplish the different stages of the procedure. Methods of this type have many disadvantages including: a great deal of skill is ordinarily required to perform the procedure, difficulty in positioning the elements of the apparatus accurately at each stage, possibility of movement of the stent as the catheter is moved back and forth, and, the amount of time required to accomplish all the stages of the procedure.

In order to minimize some of these disadvantages, delivery systems that contain stents mounted directly over the expandable section of the balloon catheter have been developed. The major technological problems that must be overcome in this type of assembly are keeping the stent from moving off the balloon during the insertion procedure and keeping the stent in a collapsed state on the balloon, and, preventing its accidental release until it is at the location where it is to be deployed. This last problem is especially critical for self-expanding stents and in cases where the device must be inserted through especially tortuous sections of the vasculature.

One approach to solving these problems has been to cover the stent and balloon with a sheath. An example of this solution is disclosed in U.S. Pat. No. 5,593,412. The sheath is withdrawn before balloon inflation by pulling the sheath in the proximal direction such that the distal end of the sheath flexes and expands and then the sheath slides over the stent and balloon. One method of reducing the amount of force needed to free the distal end of the sheath is to introduce a warm liquid to soften the material of which the sheath is manufactured. This adds time and complexity to the procedure. Pulling back to remove the sheath can cause the stent to be shifted from the desired location.

U.S. Pat. No. 4,950,227 and U.S. Pat. No. 5,108,416 illustrate another approach to solving the problem of restraining the stent during the introduction procedure. In both of these disclosures, retaining devices, in the form of sleeves or caps, are located near one or both ends of the balloon and placed over one or both ends of the stent. When the balloon is inflated, causing the stent to expand, the material of the sleeves stretches and is forced to slide backwards freeing the ends of the stent. With these systems, it is often difficult to position the center of the device exactly at the desired position because the ends are not always released simultaneously. This is an especially serious problem for self-expanding stents where the length in the contracted state is longer than that in the expanded state by an amount that typically varies by 50% to 500%.

There is thus a need for, and it would be highly advantageous to have a system and corresponding method for optimally deploying an implantable expandable intraluminal device at the required location in a body lumen.

### SUMMARY OF THE INVENTION

The present invention relates to a system and corresponding method for optimally deploying an implantable expandable intraluminal device at the required location in a body lumen.

It is thus an object of the present invention to provide a delivery system for use in positioning an implantable intraluminal device in a body lumen that overcomes the deficiencies of the prior art. It is another object of the invention to provide a method for transluminal implantation of intraluminal devices, and particularly of a braided stroke preventing device using the delivery system of the present invention. Other objects of the present invention are apparent throughout the following description.

The present invention is useful to deliver a variety of intraluminal devices and is not limited to be used with any specific device. A device which is particularly convenient to deliver with the system of the present invention is a stroke preventing device. A stroke preventing device is an implantable device that is an intravascular carotid artery stent-like device, designed specifically to prevent anterior circulation strokes from proximal embolic sources, as described, for example, in U.S. Pat. No. 6,348,063, of the same assignee hereof.

Such an implantable device is designed for positioning, inter alia, in the vicinity of a bifurcation of an artery leading to, or located in, the common carotid artery (CCA) on the one hand, and leading to a non-vital artery on the other hand. It comprises a deflecting filtering element suitable to deflect the flow of embolic material flowing toward the CCA, into the non-vital artery, while filtering the blood flowing toward the CCA. The device features a tubular body having a contracted state with a first diameter, and an expanded state having a second diameter greater than the first diameter.

In peripheral vessels, it is usually preferred to use a device that is self-expandable. A typical deflecting filter has a length of 20 mm to 150 mm and it has a diameter in the expanded state of 3 mm-30 mm (6 mm-10 mm in the carotid artery). A preferred deflecting filter comprises a braided cylindrical body.

A well known characteristic property of devices of the type to which this invention is directed is that the device elongates as it is compressed from an expanded to a contracted state, that is, the length of the device is longer in the contracted state than it is in the expanded state. Although, for purposes of illustration, self-expandable braided devices are frequently referred to in the description of the present invention, it should be clear to the man of the art that the delivery system of the present invention is in fact suitable for use with any stent or stent-like device that satisfies the requirement that its length decreases as it is expanded radially, be it braided or meshed or any other type of device.

According to one aspect of the present invention, there is provided a system for delivering and deploying an intraluminal device at a selected location in a body lumen wall, the intraluminal device being capable of assuming both a contracted condition of small diameter and long length, and an expanded condition of larger diameter and smaller length; the system comprising: an inner supporting structure for supporting the intraluminal device; an outer tube for enclosing the inner supporting structure, and the intraluminal device when supported in its contracted condition thereon, the outer tube being removable from the inner supporting structure, and the intraluminal device when supported thereon, to permit the intraluminal device to expand to its expanded condition for deployment at the selected location within the body lumen wall; and a pair of socks carried by the inner supporting structure for receiving between them the ends of the intraluminal device in its contracted condition; each the sock having: one end secured to the inner supporting structure, an opposite end facing the other sock for receiving the respective end of the intraluminal device in its contracted condition, and a length such that, when the outer tube is removed and the intraluminal device starts to expand, each sock does not release the respective end of the intraluminal device until the center region of the intraluminal device expands into engagement with the body lumen wall.

As will be more particularly described below, the inner supporting structure may be constructed to support a self-expandable intraluminal device, or it may include a balloon for expanding the intraluminal device. In the latter case, the balloon is preferably of shorter length than the intraluminal device in its contracted condition.

In one described preferred embodiment, the inner supporting structure includes an inner tube, and the one end of each sock is secured to the inner tube.

In a second described preferred embodiment, the inner supporting structure includes two coaxial inner tubes axially movable with respect to each other, the one end of each sock being secured to a different one of the inner tubes such that, by axially moving the inner tubes relative to each other, the socks may be moved towards each other at the beginning of the deployment of the intraluminal device, after removal of the outer tube, to better assure displacement and engagement of the center region of the intraluminal device with the body lumen wall before the intraluminal device is released from the socks.

In the preferred embodiment of the invention described below, a conventional catheter with balloon attached, of the type well known in the art, is used. To the catheter, there is attached a pair of socks. The purpose of these socks is to restrain the intraluminal device in its contracted position. The socks of the present invention can be made of any suitable bio-compatible material that may also be plastically or elastically deformable. In a preferred embodiment of the present invention, the material is an elastic polymer. “Bio-compatible”, in this context, means a material that can be introduced into a body cavity for the length of time needed to perform the deployment, without causing unbearable adverse effects to the subject.

The device is slipped over the balloon-catheter. The device is then radially compressed and the socks are pulled over its ends to hold it in its contracted state on the catheter. This assembly is now slipped into an outer tube, and the deployment system of the present invention is ready for insertion into the body lumen.

Once the deployment system has been guided to the desired location, the outer tube is withdrawn and inflation of the balloon is begun. As the balloon inflates, it exerts a radial force on the inside walls of the device. This causes the device to expand radially and therefore to contract in length. In a preferred embodiment of the invention, the balloon is designed such that inflation takes place from the center causing the middle of the device to come in contact with the inside wall of the lumen and anchor the device firmly in position before its ends are released from the socks. As this process continues, the ends of the device are withdrawn from the constraining socks which may or may not undergo elastic or plastic deformation, or both, during the process. Once the ends of the device are free of the socks, the device is free to expand under the influence of its internal radial force. According to a preferred embodiment of the invention, inflation of the balloon continues until the balloon presses the device against the inner wall of the lumen improving the anchoring of the device in place.

According to another aspect of the present invention, therefore, there is provided a method of utilizing the system described above for delivering and deploying the intraluminal device thereof at a selected location in a body lumen wall, comprising:

i) inserting a guiding wire and a guiding catheter through the body lumen to the selected location;

ii) utilizing the guiding wire and an imaging technique for guiding the system through the guiding catheter to the selected location in the body lumen;

iii) withdrawing the outer tube and allowing the center of the intraluminal device to expand and to come into contact with the body lumen wall, then to be released from the socks, and finally to assume a fully expanded condition pressed into the body lumen wall;

iv) covering the socks with the outer tube; and

v) withdrawing the inner tube with the socks, the outer tube, the guiding catheter, and the guiding wire, from the body lumen.

It is noted that the invention can be advantageously exploited at any suitable location in a body lumen. The present invention successfully overcomes shortcomings and limitations of presently known deployment systems used for deploying an expandable intraluminal device at the required location in a body lumen. The above, and other, characteristics, features, and, advantages, of the present invention, are better understood through the following illustrative and non-limiting detailed description of preferred embodiments thereof.

### BRIEF DESCRIPTION OF THE DRAWINGS

The invention is herein described, by way of example only, with reference to the accompanying drawings, wherein:

FIG. 1 is a schematic diagram illustrating a front view of an expandable implantable intraluminal device in the expanded state;

FIG. 2 is a schematic diagram illustrating a front view of the delivery system, the device to be deployed being in a contracted state, in accordance with the present invention;

FIG. 3A is a schematic diagram illustrating a sectional view of the deployment system in a contracted state with the outer tube in place, in accordance with the present invention;

FIG. 3B is a schematic diagram illustrating a sectional view of the deployment system just after the outer tube has been withdrawn and inflation of the balloon has begun, in accordance with the present invention;

FIG. 3C is a schematic diagram illustrating a sectional view of the deployment system just before release of the deflecting device from the sleeves, in accordance with the present invention;

FIG. 3D is a schematic diagram illustrating a sectional view of the deployment system at an intermediate state of expansion, in accordance with the present invention;

FIG. 3E is a schematic diagram illustrating a sectional view of the deployment system in a fully expanded state, in accordance with the present invention;

FIG. 3F is a schematic diagram illustrating a sectional view of the deployment system after deployment of the device and deflation of the balloon, in accordance with the present invention;

FIG. 4A is a schematic diagram illustrating a view of the embodiment of the invention including a middle tube in a contracted state, in accordance with the present invention;

FIG. 4B is a schematic diagram illustrating a view of the embodiment of the invention including a middle tube just after the outer tube has been withdrawn and inflation of the balloon has begun, in accordance with the present invention; and

FIG. 5 is a schematic diagram illustrating a view of the proximal end of the delivery system for the embodiment including a middle tube. in accordance with the present invention.

### DESCRIPTION OF THE PREFERRED EMBODIMENTS

The present invention relates to a system and corresponding method for optimally deploying an implantable expandable intraluminal device at the required location in a body lumen.

It is to be understood that the invention is not limited in its application to the details of the order or sequence of steps of operation or implementation of the method, or, to the details of construction, arrangement, and, composition of the components of the deployment system, set forth in the following description, drawings, or examples. For example, the present invention can be advantageously exploited at any suitable location in a body lumen. The present invention is capable of other embodiments or of being practiced or carried out in various ways. Also, it is to be understood that the phraseology and terminology employed herein are for the purpose of description and should not be regarded as limiting.

Components, steps, operation, and implementation of a system and corresponding method for optimally deploying an implantable expandable intraluminal device at the required location in a body lumen, according to the present invention are better understood with reference to the following description and accompanying drawings. Throughout the following description and accompanying drawings, like reference numbers refer to like elements.

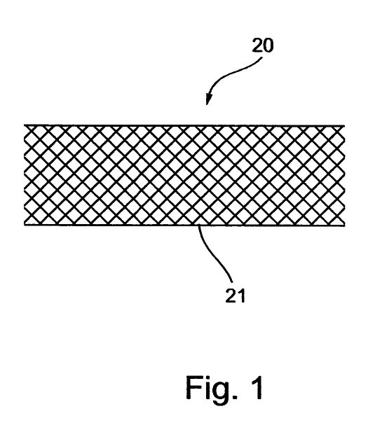

Referring now to the drawings, FIG. 1 is a schematic diagram illustrating a front view of an expandable implantable intraluminal device in expanded state. The intraluminal device features a substantially tubular body 20, which has been formed according to any technique known in the art, for example by braiding filaments 21 to form a braided tubular body. Since precise details of the construction of the device are not relevant to an understanding of the present invention, they are not addressed here for the sake of brevity. A further requirement for use with the deployment system of the present invention, is that the deflecting device of FIG. 1 has a length in its contracted state that is longer than its length in its expanded state.

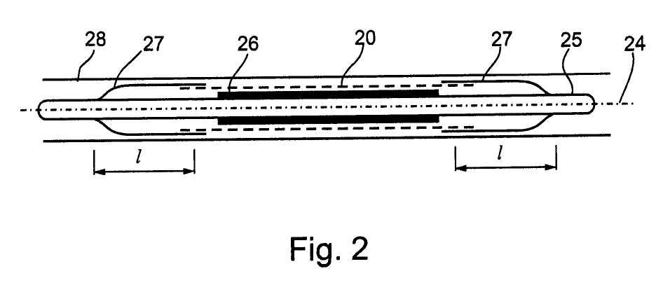

FIG. 2 shows the delivery system of the present invention. A conventional balloon-catheter 25 is placed on a guiding wire 24. The guiding wire and guiding catheter (not shown in the figures) are not novel per se. They are standard elements used for translumenal introduction of medical devices and are not described further since they are well known to persons skilled in the art. The balloon is shown at 26 in the figure. An implantable device 20 (dashed lines in the figure) is then placed over the balloon on the catheter. A pair of socks 27 is placed over the catheter. Each sock of the pair is located such that it can be placed over one end of the deflecting device and will hold the device onto the catheter when the device is in its collapsed state. Finally, the catheter is slipped into an outer tube 28 and the assembled device is ready for introduction into the body lumen.

The outer tube has several functions. Firstly, in situations in which a guiding catheter is not present, it serves to protect the walls of the body lumen from damage by the device as it is inserted and withdrawn from the implantation site. Secondly, it serves to protect the device from mechanical damage during the insertion procedure. Finally, and most importantly, the outer tube is a safety device that prevents premature expansion of the deployable device. Accidental release of the device can occur, for example, as a result of a mistake on the part of a member of the surgical team, or as a result of the deformation of the device as it is introduced through curved parts of the body lumen causing an end of the device to be pulled out of it's restraining sock.

The socks can be made from any suitable bio-compatible material. In a preferred embodiment of the invention the socks are made of an elastic polymer, such as nylon, Pebax, Peek, or PE. The socks are held in place on the catheter by an appropriate technique, such as welding or by the use of a suitable glue. The socks, according to the particular preferred embodiment of FIG. 2, are manufactured such that the depth of the portion of the sock that is not attached to the catheter, corresponding to the length l (FIG. 2), is appropriate for proper functioning of the device as described.

The balloon is chosen such that its length is slightly shorter than the length of the deflecting device in its collapsed state. Choosing the length of the balloon in this manner guarantees that the ends of the balloon will be clear of the ends of the deflecting device and of the socks, and therefore will not interfere with the release of the deflecting device. In a preferred embodiment of the invention, the balloon is constructed such that the process of inflation begins at the center and advances symmetrically towards both of its ends.

The diameter of the self-expanding device may somewhat vary for different applications. However, the diameter in the closed state is up to about 3 mm, while when expanded, the diameter may vary in the range of up to 30 mm. The devices are typically 20 mm to 150 mm long in their expanded state. The considerations necessary for choosing the appropriate dimensions of the device and thus of the balloon and catheter are well known to the man of the art and, therefore will not be discussed here.

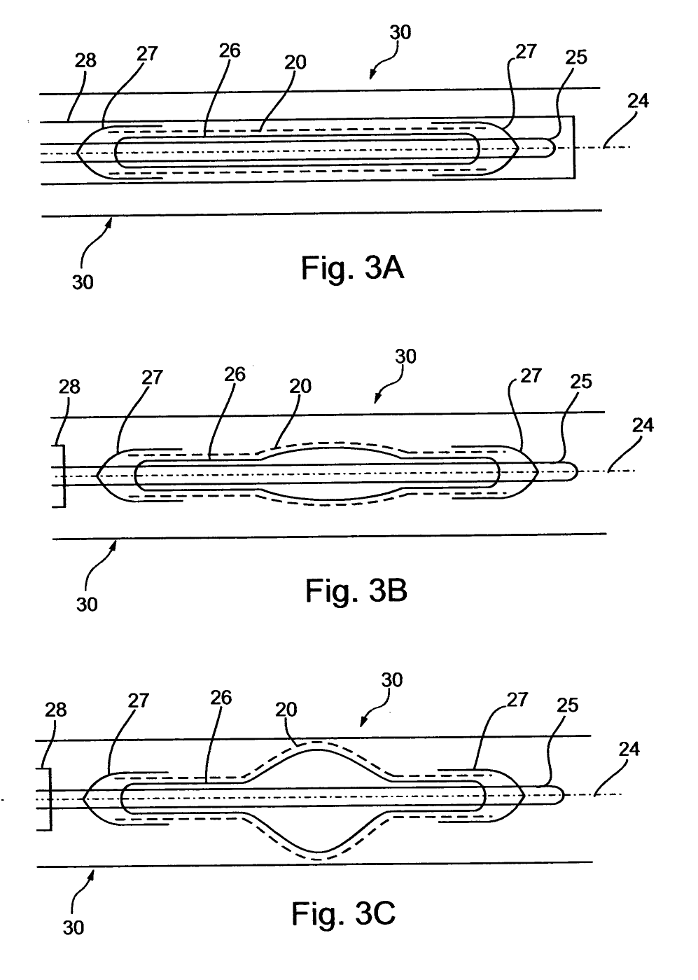

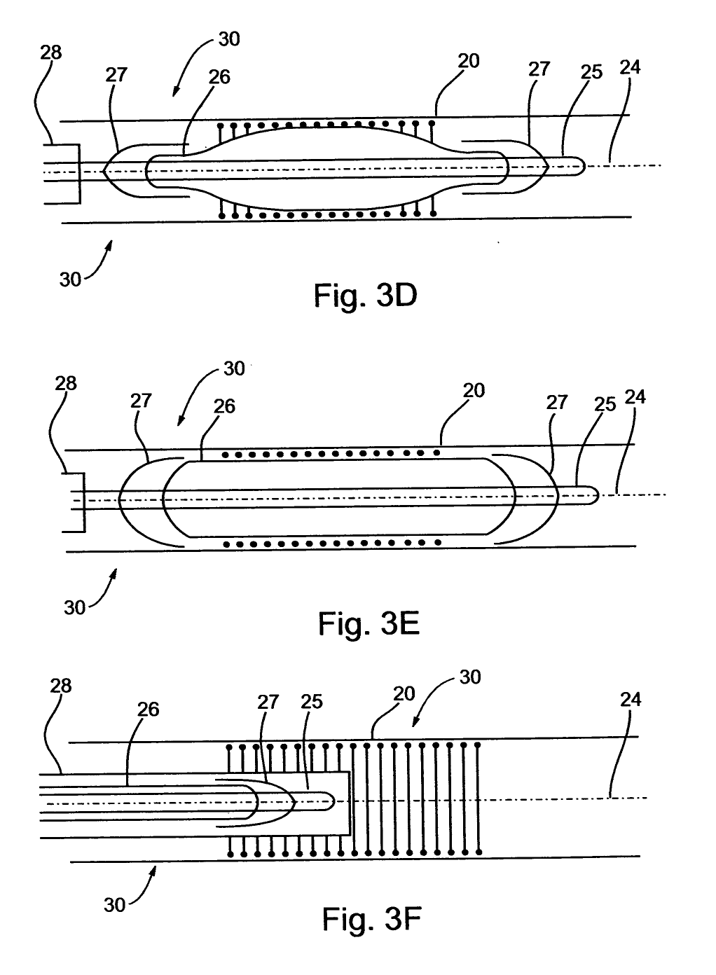

FIGS. 3A-3F show the steps in the release and deployment of the self-expanding device. In order to simplify the drawings, cross sections in the plane of the longitudinal axis of the catheter are shown in the figures. In FIG. 3A through FIG. 3F, the elements of the figure are identified as follows: 20 is the implantable device; 24 is the guiding wire; 25 is the balloon-catheter; 26 is the balloon; 27 is a sock; 28 is the outer tube; and 30 is the wall of the lumen.

FIG. 3A shows the system in compressed form, during insertion through the body lumen. The self-expanding device is placed over the balloon-catheter, and its radial dimension is reduced until it is in contact with the collapsed balloon. Reducing the radius results in an elongation of the device along the axis of the catheter. Typically the length of the device in its collapsed state is 50% to 500% longer than its length in its expanded state. The device is collapsed onto the balloon, the two sleeves that are attached to the balloon-catheter are placed over the respective ends of the device, and an outer tube is slipped over the entire assembly.

FIG. 3B shows the situation after the device has reached the location in the body lumen where it will be released. As shown in FIG. 3B, the outer tube has been pulled backwards and the inflation of the balloon has begun.

FIG. 3C shows the preferred embodiment of the invention in which the balloon expands from the center outwards. The balloon inflates from the middle, causing the device to expand until it reaches the wall of the lumen. In order to release the device, the balloon is inflated using techniques well known in the art. As the balloon begins to inflate, it exerts a radial force on the device. As the radius of the device increases, its length decreases pulling its ends out of the socks. During this stage the deflecting device is in contact with the balloon. The length of the socks has been determined using the known properties of the expandable device and the diameter of the lumen, so that the ends of the device are not released until the center of the device is firmly anchored to the walls of the lumen by its elastic forces as well as the balloon.

FIG. 3D shows as intermediate stage in the deployment of the device. In the preferred embodiment shown here, elastic forces cause the released device to expand rapidly until it comes in contact with the inside wall of the body lumen. At the same time, the balloon continues to expand but at a rate of expansion slower than that of the device. At this stage the device is no longer in contact with the balloon and comes gently into contact with the wall of the lumen under the influence of its own elastic forces only.

FIG. 3E shows the final stage of the deployment. Here the balloon is fully expanded pressing the wall of the device against the inner wall of the body lumen. The pressure exerted by the balloon is necessary to fully expand the device if a local calcified area of the lumen has prevented a small section of the device from expanding. The pressure exerted by the balloon also insures that the device firmly contacts the walls of the lumen. Such contact causes a proliferation of cells through the net of the device, and strongly anchors it to the lumen thus preventing its accidental displacement. The physiological processes leading to such anchoring are well known in the art, and will therefore not be discussed herein in detail, for the sake of brevity.

FIG. 3F shows the situation at the start of the withdrawal of the delivery system. The outer tube has been pushed back over the socks and balloon which have collapsed onto the inner tube. The balloon has been deflated in a conventional manner by drawing out the fluid used for inflation. The inner and outer tubes, with attached socks and balloon are then withdrawn through the guiding catheter leaving behind the self-expanding device.

Now the method for placement and deployment of the system of the invention will be described. First the guiding wire followed by a guiding catheter is introduced through the vasculature of a subject as in any conventional procedure of this kind. Then, the balloon-catheter, with self-expanding device mounted on it and held in a collapsed form by the socks of the invention, as described above with reference to FIG. 2, and covered by an overtube is placed over the guiding wire and inserted through the guiding catheter until the device is in the proper position. In order to properly guide and locate the device, radio opaque markers may be required. These markers can be supplied on the catheter or on the device or both. Since the techniques of supplying and using radio opaque markers are well known to the man of the art, they are not shown in the figures or discussed here.

If the implantable device is a deflecting device used to prevent the occurrence of strokes, it can be placed in several different locations in the body depending on the clinical indications of each case. A typical, but not limiting, location for the device is the bifurcation junction of the carotid artery. In this case, the deflecting device is positioned within the bifurcation zone opposite the inlet to the internal carotid artery (ICA). The body of the deflecting device is anchored against the respective inner walls of the common carotid artery (CCA) and the external carotid artery (ECA), respectively. In this position, embolic material in blood flowing into the CCA and, contacts the deflecting member, and is prevented from entering the ICA and is thus deflected into the ECA.

Once the device is located at the desired location, the outer tube is withdrawn and expansion of the balloon is begun. The device is released from the restraining socks, allowed to expand, and pressed firmly against the walls of the body lumen as described above with reference to FIGS. 3B through 3E. It should be noted that, during the release phase of the process of deployment, the change in length takes place simultaneously and symmetrically at both ends of the deflecting device. Thus, both ends of the device move out of the socks at the same time and, even in situations in which the balloon does not succeed in anchoring the center of the device before it is released as described above, the device expands without moving laterally on the balloon-catheter. This fact greatly simplifies and increases the accuracy of the positioning of the device.

After deployment of the deflecting device is completed, the balloon is deflated and then the outer tube is pushed back over the balloon and socks before withdrawing the assembly from the subject as in any other similar procedure. Since the material of which the socks are manufactured is slightly elastic, they are held tightly on the surface of the catheter after the release of the device and thus can cause no damage to the walls of the vasculature when the catheter is withdrawn, even in cases in which a guiding catheter and an outer tube are not present.

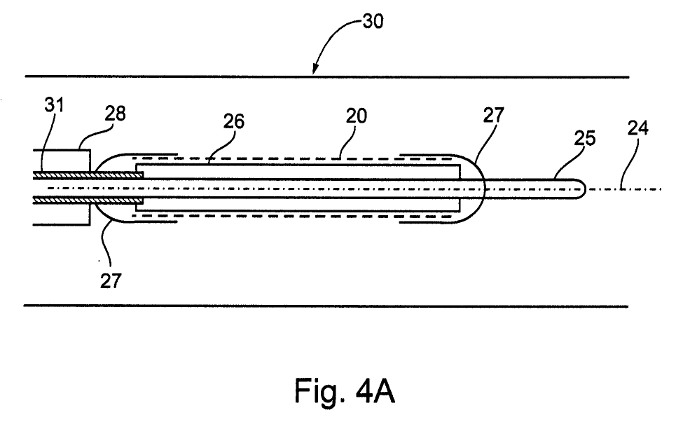

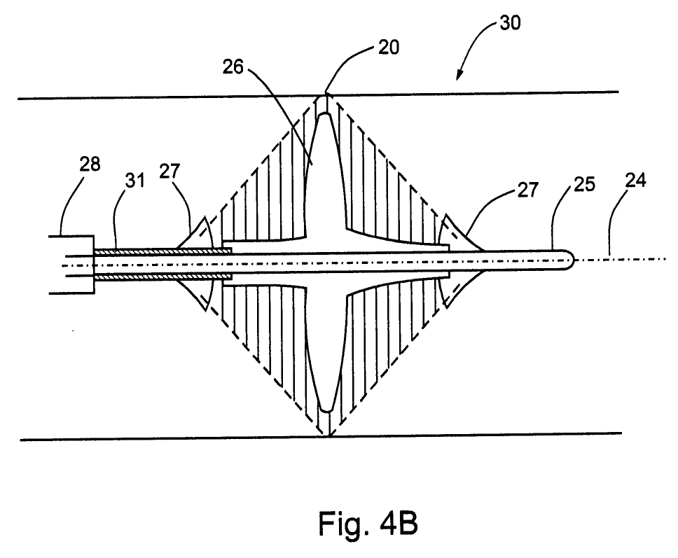

In FIG. 4A there is schematically shown a delivery system according to another embodiment of the invention. In FIG. 4A, the delivery system is shown as it would appear at the site where the device is to be implanted. The outer tube 28 has been withdrawn exposing the expandable device 20 collapsed on the balloon 26 and inner tube 25 and held in place by a pair of socks 27. The wall of the lumen is indicated by numeral 30, the guiding wire is numeral 24, and the guiding catheter is not shown. In this embodiment, an additional element, that is not present in the previously described embodiments of the invention, is a middle tube 31. The middle tube slides over the inner tube and inside the outer tube. The distal sock and end of the balloon are held in place on the inner tube and the proximal sock and end of the balloon are attached to the middle tube.

FIG. 4B schematically shows the first stage in the deployment of the device of FIG. 4A. In this situation, the middle tube is moved relative to the inner tube such that the socks are moved closer together. As their distal and proximal ends are forced together, the middle of the device and of the balloon are pushed towards the wall of the lumen. From this position, the balloon has to be inflated much less than in the previously described embodiment of the invention in order to anchor the middle of the device against the wall of the lumen. Once the middle of the device is anchored in place the inflation continues and the process proceeds substantially as described above with reference to FIGS. 3D-3E. In some situations, during the expansion of the balloon and device, the middle tube may have to be allowed to move relative to the inner tube in order to allow the release of the ends of the device from the socks.

This embodiment of the invention has some advantages over the delivery system previously described with reference to FIGS. 2 and 3A to 3F, especially in cases in which the expandable device is to be implanted in a body lumen of large diameter. Creating the first step of the expansion in this manner helps to guarantee that the device is implanted from the middle first, thereby reducing the chance of motion of the device during the remainder of the process. In addition, in order for the balloon of the previously described embodiment of the invention to satisfy the dual requirements of being able to expand to a large diameter and also compress to a small diameter for insertion and withdrawal from the lumen, it must necessarily have thin walls. In the embodiment shown in FIGS. 4A and 4B, a thicker walled balloon can be employed.

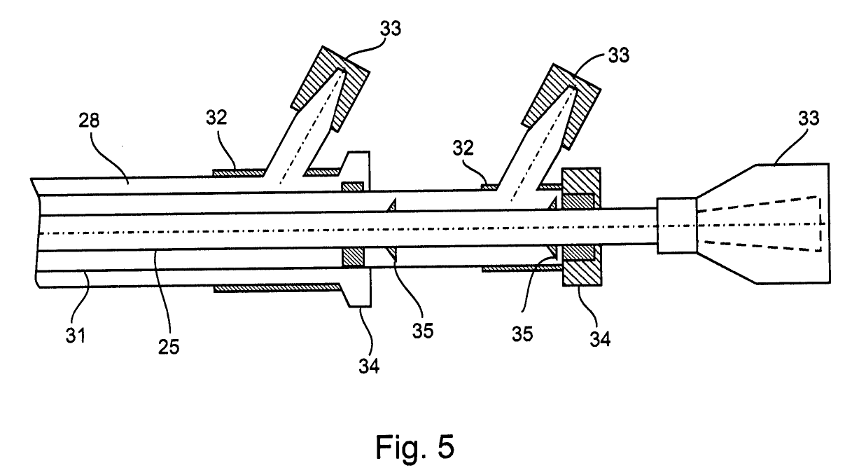

The method for placement and deployment of the system of this embodiment of the invention is essentially the same as that described previously, with the exception of the first step in the inflation of the balloon as described above and that, after deployment of the expandable device, the middle tube and inner tube must be moved relative to each other prior to withdrawal of the system. FIG. 5 schematically shows one possible locking arrangement for the delivery system portrayed in FIG. 4A. FIG. 5 represents the proximal end of the delivery system that is located outside of the body of the subject. As in FIG. 4A, the inner, middle, and, outer, tubes are designated by the numerals 25, 31, and 28 respectively. The rest of the components shown in FIG. 5 are standard and well known to persons skilled in the art, and therefore neither they nor the methods of employing them will be further described here. The numeral 32 designates a standard Y-connector with locking mechanism 34. Numeral 33 depicts a Luer lock and 35 depicts stoppers on the inner tube.

To deploy the implantable device using the configuration shown in FIG. 5, after the device reaches the desired position in the body, the middle tube is locked to the outer tube and then the inner tube is pulled proximally relative to the middle tube as the first step in the expansion of the balloon and device. It will be appreciated by the skilled person, that many other arrangements of the standard components (for example, locking the inner and outer tubes together and moving the middle tube relative to them) are possible, depending on the requirements of the medical procedure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A system for delivering and for deploying an intraluminal device at a selected location in a body lumen wall, said intraluminal device being capable of assuming both a contracted condition of small diameter and long length, and an expanded condition of larger diameter and smaller length; said system comprising:
an inner supporting structure for supporting the intraluminal device;
an outer tube for enclosing said inner supporting structure, and the intraluminal device when supported in its contracted condition thereon, said outer tube being removable from the inner supporting structure, and the intraluminal device when supported thereon, to permit the intraluminal device to expand to its expanded condition for deployment at said selected location within the body lumen wall;
and a pair of socks carried by said inner supporting structure for receiving between them the ends of said intraluminal device in its contracted condition;
each said sock having: one end secured to said inner supporting structure, an opposite end facing the other sock for receiving the respective end of the intraluminal device in its contracted condition, and a length such that, when the outer tube is removed and the intraluminal device starts to expand, each sock does not release the respective end of the intraluminal device until the center region of the intraluminal device expands into engagement with said body lumen wall.

2. The system according to claim 1, wherein said inner supporting structure is constructed to support a self-expandable intraluminal device.

3. The system according to claim 1, wherein said inner supporting structure includes a balloon for expanding said intraluminal device.

4. The system according to claim 3, wherein said balloon is of shorter length than said intraluminal device in its contracted condition.

5. The system according to claim 1, wherein said inner supporting structure includes an inner tube, and said one end of each sock is secured to said inner tube.

6. The system according to claim 1, wherein said inner supporting structure includes two coaxial inner tubes axially movable with respect to each other, said one end of each sock being secured to a different one of said inner tubes such that, by axially moving the inner tubes relative to each other, said socks may be moved towards each other at the beginning of the deployment of the intraluminal device, after removal of the outer tube, to better assure displacement and engagement of the center region of the intraluminal device with said body lumen wall before the intraluminal device is released from said socks.

7. The system according to claim 1, wherein said socks are made of a polymer having some elasticity.

8. The system according to claim 1, in combination with an expandable intraluminal device supported on said inner supporting structure.

9. The system according to claim 8, wherein said intraluminal device is self-expandable.

10. The system according to claim 8, wherein said inner supporting structure includes a balloon for expanding said intraluminal device.

11. The system according to claim 10, wherein said balloon is of shorter length than said intraluminal device in its contracted condition.

12. The system according to claim 8, wherein said inner supporting structure includes an inner tube, and said one end of each sock is secured to said inner tube.

13. The system according to claim 8, wherein said inner supporting structure includes two coaxial inner tubes axially movable with respect to each other, said one end of each sock being secured to a different one of said inner tubes such that, by axially moving the inner tubes relative to each other, said socks may be moved towards each other at the beginning of the deployment of the intraluminal device, after removal of the outer tube, to better assure displacement and engagement of the center region of the intraluminal device with said body lumen wall before the intraluminal device is released from said socks.

14. The system according to claim 8, wherein said intraluminal device includes a braided tube.

15. A method of utilizing the system of claim 8 for delivering and deploying the intraluminal device thereof at a selected location in a body lumen wall, comprising:
vi) inserting a guiding wire and a guiding catheter through the body lumen to said selected location;
vii) utilizing said guiding wire and an imaging technique for guiding said system through said guiding catheter to the selected location in said body lumen;
viii) withdrawing said outer tube and allowing the center of said intraluminal device to expand and to come into contact with the body lumen wall, then to be released from said socks, and finally to assume a fully expanded condition pressed into the body lumen wall;
ix) covering said socks with said outer tube; and
x) withdrawing said inner tube with said socks, said outer tube, said guiding catheter, and said guiding wire, from said body lumen.

16. The method according to claim 15, wherein said intraluminal device is self-expandable.

17. The method according to claim 15, wherein said step of withdrawing said outer tube and allowing the center of said intraluminal device to expand further comprises expanding a balloon carried by said inner supporting structure thereby urging the center of said intraluminal device to expand.

18. The method according to claim 17, wherein said balloon is of shorter length than said intraluminal device in its contracted condition.

19. The method according to claim 15, wherein said inner supporting structure includes an inner tube, and said one end of each sock is secured to said inner tube.

20. The method according to claim 15, wherein said inner supporting structure includes two coaxial inner tubes axially movable with respect to each other, said one end of each sock being secured to a different one of said inner tubes, and wherein said step of withdrawing said outer tube and allowing the center of said intraluminal device to expand further comprises axially moving the inner tubes relative to each other, thereby moving said socks towards each other at the beginning of the deployment of the intraluminal device, after removal of the outer tube, thus better assuring displacement and engagement of the center region of the intraluminal device with said body lumen wall before the intraluminal device is released from said socks.
